(19) United States
(12) Patent Application Publication
(10) Pub. No.: US 2005/0283197 A1
(43) Pub. Date: Dec. 22, 2005

(54) Systems and methods for hypotension

(76) Inventors: Douglas R. Daum, Woodbury, MN (US); Avram Scheiner, Vadnais Heights, MN (US)

Correspondence Address: SCHWEGMAN, LUNDBERG, WOESSNER & KLUTH, 1600 TCF TOWER, 121 SOUTH EIGHT STREET, MINNEAPOLIS, MN, 55402, US

(21) Appl. No.: 11/141,260

(22) Filed: May 31, 2005

Related U.S. Application Data
(63) Continuation-in-part of application No. 09/879,665, filed on Jun. 12, 2001, now Pat. No. 6,907,288.
Application No. 09/879,665 is a continuation-in-part of application No. 09/832,365, filed on Apr. 10, 2001, now Pat. No. 6,912,420.

(51) Int. Cl. A61N 1/365

(52) U.S. Cl. 607/17; 607/3; 607/9

(57) ABSTRACT

This document discusses, among other things, systems and methods that detect hypotension based on a measurement of thoracic impedance. It also provides an alert, a logging, or a therapy to treat the hypotension. Examples of anti-hypotension therapies include, among other things, pacing therapy, neural stimulation therapy, drug infusion therapy, or gene therapy.

### CROSS-REFERENCE TO RELATED APPLICATIONS

This patent application is a continuation-in-part of Daum U.S. patent application Ser. No. 09/879,665, filed on Jun. 12, 2001, entitled “CARDIAC RHYTHM MANAGEMENT SYSTEM ADJUSTING RATE RESPONSE FACTOR FOR TREATING HYPOTENSION,” and assigned to Cardiac Pacemakers, Inc., which, in turn, is a continuation-in-part of Scheiner et al. U.S. patent application Ser. No. 09/832,365, filed on Apr. 10, 2001, entitled “CARDIAC RHYTHM MANAGEMENT SYSTEM FOR HYPOTENSION,” and assigned to Cardiac Pacemakers, Inc., and the disclosure of each of the above referenced patent applications is incorporated herein by reference in its entirety.

This patent application is also related to Libbus et al. U.S. patent application Ser. No. 11/124,791 (Attorney Docket 279.780US1), entitled METHOD AND APPARATUS FOR CONTROLLING AUTONOMIC BALANCE USING NEURAL STIMULATION, filed on May 9, 2005, which is assigned to Cardiac Pacemakers, Inc., and which is incorporated herein by reference in its entirety.

This patent application is also related to Girouard et al. U.S. patent application Ser. No. 10/788,906 (Attorney Docket No. 279.696US1) entitled METHOD AND APPARATUS FOR DEVICE CONTROLLED GENE EXPRESSION, filed on Feb. 27, 2004, and which is assigned to Cardiac Pacemakers, Inc., and which is incorporated herein by reference in its entirety.

### TECHNICAL FIELD

This patent document pertains generally to medical devices and methods, and more particularly, but not by way of limitation, to systems and methods for treating hypotension.

### BACKGROUND

When functioning properly, the human heart maintains its own intrinsic rhythm, and is capable of pumping adequate blood throughout the body's circulatory system. However, some people have irregular cardiac rhythms, referred to as cardiac arrhythmias. Such arrhythmias result in diminished blood circulation. One mode of treating cardiac arrhythmias uses drug therapy. Drugs are often effective at restoring normal heart rhythms. However, drug therapy is not always effective for treating arrhythmias of certain patients. For such patients, an alternative mode of treatment is needed. One such alternative mode of treatment includes the use of a cardiac rhythm management system. Such systems are often implanted in the patient and deliver therapy to the heart.

Cardiac rhythm management systems include, among other things, pacemakers, also referred to as pacers. Pacers deliver timed sequences of low energy electrical stimuli, called pace pulses, to the heart, such as via an intravascular leadwire or catheter (referred to as a “lead”) having one or more electrodes disposed in or about the heart. Heart contractions are initiated in response to such pace pulses (this is referred to as “capturing” the heart). By properly timing the delivery of pace pulses, the heart can be induced to contract in proper rhythm, greatly improving its efficiency as a pump. Pacers are often used to treat patients with bradyarrhythmias, that is, hearts that beat too slowly, or irregularly. Such pacers coordinate atrial and ventricular contractions to improve pumping efficiency. Cardiac rhythm management systems also include coordination devices for coordinating the contractions of both the right and left sides of the heart for improved pumping efficiency.

Cardiac rhythm management systems also include defibrillators that are capable of delivering higher energy electrical stimuli to the heart. Such defibrillators also include cardioverters, which synchronize the delivery of such stimuli to portions of sensed intrinsic heart activity signals. Defibrillators are often used to treat patients with tachyarrhythmias, that is, hearts that beat too quickly. Such too-fast heart rhythms also cause diminished blood circulation because the heart isn't allowed sufficient time to fill with blood before contracting to expel the blood. Such pumping by the heart is inefficient. A defibrillator is capable of delivering an high energy electrical stimulus that is sometimes referred to as a defibrillation countershock, also referred to simply as a “shock.” The countershock interrupts the tachyarrhythmia, allowing the heart to reestablish a normal rhythm for the efficient pumping of blood. In addition to pacers, cardiac rhythm management systems also include, among other things, pacer/defibrillators that combine the functions of pacers and defibrillators, drug delivery devices, and any other implantable or external systems or devices for diagnosing or treating cardiac arrhythmias.

One problem faced by some patients is hypotension, that is, low blood pressure. Hypotension can result in dizziness, sometimes referred to as presyncope. Hypotension can even lead to unconsciousness, sometimes referred to as syncope. One cause of hypotension is an excess shifting of blood in the circulatory system toward the extremities (arms and legs) and away from vital organs in the patient's head and thorax. This can occur, for example, when the patient changes posture from lying horizontal or sitting with legs elevated to a position in which the patient is sitting or standing erect. Hypotension resulting from such changes in posture is referred to herein as orthostatic hypotension. However, hypotension may also have causes other than changes in posture. For example, maintaining the same posture for an extended period of time (e.g., sitting erect during an intercontinental airplane flight) may also cause hypotension. Moreover, certain cardiovascular disorders may result in hypotension independent of postural changes, or may exacerbate orthostatic hypotension.

For example, disautonomic syncope is a problem with the autonomic nervous system. In normal patients, the autonomic nervous system constricts the blood vessels in the extremities in response to a change to a more upright posture. This venoconstriction of the blood vessels in the extremities reduces the amount of blood that would otherwise shift to the extremities when the patient changes to a more upright posture. In some patients, however, this response by the autonomic nervous system is absent, or is even reversed by a venodilation of blood vessels in the extremities. Such patients are likely to experience hypotension. Moreover, this deficient response by the autonomic nervous system may occur even without changes in posture, leading to hypotension that is not necessarily orthostatic in nature.

Another example of a cardiovascular cause of hypotension is vasovagal syncope. In normal patients, a change to a more upright posture results in an increased heart rate. For example, for a patient that is at rest, the heart rate may temporarily increase from 60 beats per minute (bpm) to 80 bpm when the patient stands up after laying horizontally. In some patients, however, this autonomic response is absent-resulting in a drop in heart rate. This may also lead to hypotension as blood shifts away from the head and thorax into the extremities. Regardless of the cause of hypotension, the resulting symptoms of dizziness or loss of consciousness may be extremely dangerous. This is particularly so for elderly patients who are at increased risk of injury from a fall resulting from the dizziness or loss of consciousness. Hypotension is also an obvious danger for persons operating motor vehicles or other machinery. Therefore, hypotension presents undesirable symptoms and associated risks.

### BRIEF DESCRIPTION OF THE DRAWINGS

In the drawings, which are not necessarily drawn to scale, like numerals describe substantially similar components throughout the several views. Like numerals having different letter suffixes represent different instances of substantially similar components. The drawings illustrate generally, by way of example, but not by way of limitation, various embodiments discussed in the present document.

FIG. 1 is a schematic/block diagram illustrating generally one embodiment of portions of a cardiac rhythm management system.

FIG. 2 is a block diagram illustrating generally one embodiment of portions of a signal processor associated with detecting hypotension based on thoracic impedance.

FIG. 3 is a schematic/block diagram illustrating generally, one embodiment of portions of a hypotension detection module.

FIG. 4 is a schematic/block diagram illustrating generally, one embodiment of portions of a hypotension detection module.

FIG. 5 is a block diagram illustrating generally one embodiment of portions of a blending module providing an indicated pacing rate based on activity, breathing, and/or a thoracic impedance-based measurement of hypotension.

FIG. 6 is a schematic illustration of an alternate electrode arrangement including one or more defibrillation electrodes.

FIG. 7 is a schematic illustration of an alternate electrode arrangement including one or more atrial electrodes.

FIG. 8 is a schematic illustration of an alternate electrode arrangement including one or more left heart electrodes such as, for example, left ventricular electrodes.

FIG. 9 is a schematic illustration of an alternate electrode arrangement for measuring a component of thoracic impedance between atrial electrodes and ventricular electrodes.

FIG. 10 is a schematic illustration of an alternate electrode arrangement for measuring a component of thoracic impedance between right heart (e.g., right ventricular) and left heart (e.g., left ventricular) electrodes.

FIG. 11 is a schematic/block diagram example of portions of a cardiac rhythm management system and portions of an environment in which it is used.

FIG. 12 is a block diagram example of a hypotension detection circuit using an activity sensing circuit.

FIG. 13 is a graph example of one technique for determining the indicated pacing rate from the sensor-indicated metabolic need.

FIG. 14 is a block diagram illustrating generally portions of an implantable medical device that receives a thoracic impedance signal from electrodes associated with a portion of a subject's thorax, and delivers a therapy or alert responsive to hypotension.

### DETAILED DESCRIPTION

The following detailed description includes references to the accompanying drawings, which form a part of the detailed description. The drawings show, by way of illustration, specific embodiments in which the invention may be practiced. These embodiments, which are also referred to herein as “examples,” are described in enough detail to enable those skilled in the art to practice the invention. The embodiments may be combined, other embodiments may be utilized, or structural, logical and electrical changes may be made without departing from the scope of the present invention. The following detailed description is, therefore, not to be taken in a limiting sense, and the scope of the present invention is defined by the appended claims and their equivalents.

In this document, the terms “a” or “an” are used, as is common in patent documents, to include one or more than one. In this document, the term “or” is used to refer to a nonexclusive or, unless otherwise indicated. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

The present methods and apparatus will be discussed in applications involving implantable medical devices including, but not limited to, implantable cardiac rhythm management systems such as pacemakers, cardioverter/defibrillators, pacer/defibrillators, and biventricular or other multi-site coordination devices. However, it is understood that the present methods and apparatus may be employed in unimplanted devices, including, but not limited to, external pacemakers, cardioverter/defibrillators, pacer/defibrillators, biventricular or other multi-site coordination devices, monitors, programmers and recorders.

This document discusses a cardiac rhythm management system that detects hypotension by measuring an impedance across a portion of a patient's thorax, referred to as “transthoracic impedance” or simply abbreviated as “thoracic impedance.” In this document, the term thorax is understood to include the portion of the subject's body other than the head, arms, and legs. The system provides appropriate responsive therapy, such as by adjusting the rate of delivery of pacing stimuli to the heart to help avoid symptoms associated with hypotension, such as dizziness or fainting.

### EXAMPLE 1

### Electrode Configuration and Top-Level Block Diagram

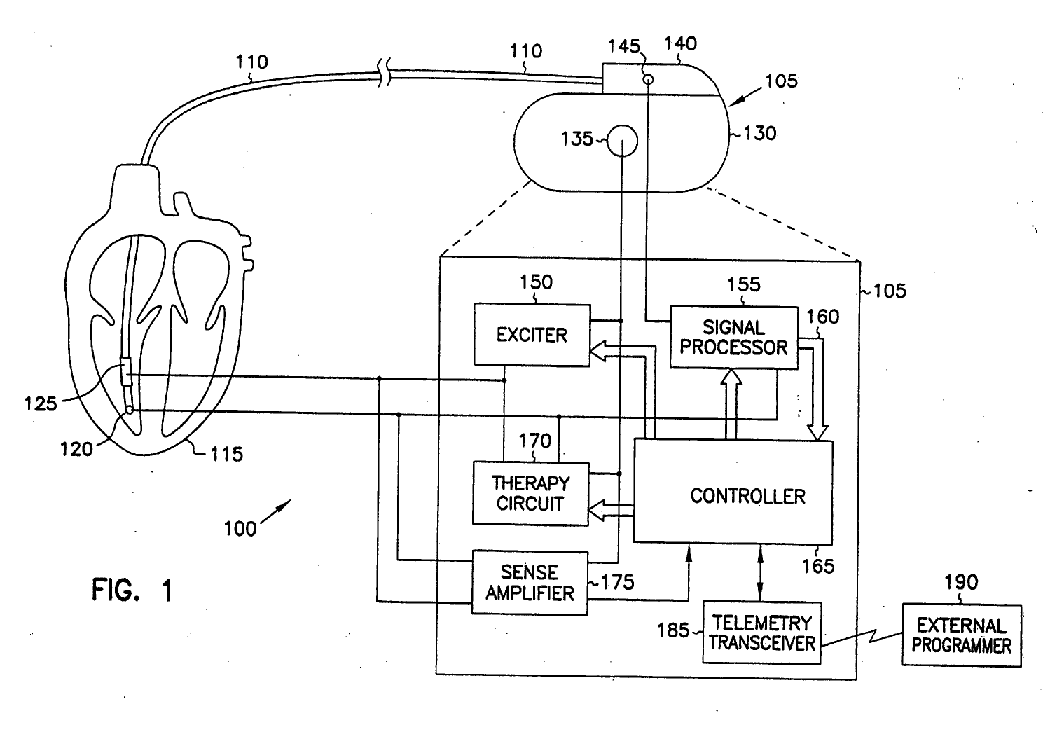

FIG. 1 is a schematic/block diagram illustrating generally, by way of example, but not by way of limitation, one embodiment of a cardiac rhythm management system 100 according to the present invention. In this embodiment, system 100 includes, among other things, cardiac rhythm management device 105 and leadwire (“lead”) 110 for communicating signals between device 105 and a portion of a living organism, such as heart 115. Embodiments of device 105 include, among other things, bradycardia and antitachycardia pacemakers, cardioverters, defibrillators, combination pacemaker/defibrillators, drug delivery devices, and any other implantable or external cardiac rhythm management apparatus capable of providing therapy to heart 115. System 100 may also include additional components such as, for example, a remote programmer 190 capable of communicating with device 105 via a transmitter or receiver, such as telemetry transceiver 185.

In one embodiment, portions of system 100 (e.g., device 105) are implantable in the living organism, such as in a pectoral or abdominal region of a human patient, or elsewhere. In another embodiment, portions of system 100 (e.g., device 105) are alternatively disposed externally to the human patient. In the illustrated embodiment, portions of lead 110 are disposed in the right ventricle, however, any other positioning of lead 110 is included within the present invention. For example, lead 110 may alternatively be positioned in a location that is associated with the right atrium and/or superior vena cava, the coronary sinus or great cardiac vein, the left atrium or ventricle, epicardially, or elsewhere. In one embodiment, lead 110 is a commercially available bipolar pacing lead. System 100 can also include other leads in addition to or instead of lead 110, appropriately disposed, such as in or around heart 115, or elsewhere. In one external embodiment, lead 110 is disposed external to the patient and includes external skin electrodes that are associated with the heart and/or thorax.

In one embodiment, system 100 includes at least four electrodes, such as discussed in Hauck et al. U.S. Pat. No. 5,284,136 entitled “DUAL INDIFFERENT ELECTRODE PACEMAKER,” assigned to Cardiac Pacemakers, Inc., the disclosure of which is incorporated herein by reference in its entirety. It is understood, however, that the present invention also includes using a different number of electrodes (e.g., 2 or 3 electrodes, or more than 4 electrodes). In one example, a first conductor of multiconductor lead 110 electrically couples a first electrode, such as tip electrode 120 (e.g., disposed at the apex of the right ventricle of heart 115), to device 105. A second conductor of multiconductor lead 110 independently electrically couples a second electrode, such as ring electrode 125, to device 105. In one embodiment, device 105 includes a hermetically sealed housing 130, formed from a conductive metal, such as titanium. Housing 130 (also referred to as a “case” or “can”) is substantially covered over its entire surface by a suitable insulator, such as silicone rubber, except for at a window that forms a third electrode, referred to as a “case” or “can” or “housing” electrode 135. In one embodiment, a header 140 is mounted on housing 130 for receiving lead 110. Header 140 is formed of an insulative material, such as molded plastic. In the illustrated embodiment, header 140 also includes at least one receptacle, such as for receiving lead 110 and electrically coupling conductors of lead 110 to device 105. Header 140 also includes a fourth electrode, referred to as indifferent electrode 145.

FIG. 1 also illustrates generally portions of device 105, together with schematic illustrations of connections to the various electrodes. Device 105 includes an electrical energy source serving as a test signal generator, such as exciter 150. Exciter 150 delivers an electrical test energy, such as a strobed sequence of current pulses or other test energy, to heart 115 (e.g., between ring electrode 125 and tip electrode 120, or using any other electrode configuration suitable for delivering the current pulses). In response to the excitation signal provided by exciter 150, a response signal is sensed by signal processor 155 (e.g., between tip electrode 120 and indifferent electrode 145, or using any other suitable electrode configuration). The electrical excitation signal is also referred to herein as a test signal or test stimulus because it serves as an active energy source from which a heart impedance is detected; however, it is understood that this signal need not, and typically does not, have the magnitude of energy required to excite or stimulate cardiac or other muscular tissue to contract. Instead, the excitation signal need only provide enough response for the signal processor to obtain useful information about heart impedance. Moreover, while the illustrated embodiment uses a current excitation stimulus to obtain a voltage response signal from which heart impedance can be determined, it is understood that a voltage excitation stimulus and current response signal could also be used to obtain heart impedance information. One example of using a high frequency carrier signal to provide a test stimulus and obtain a thoracic impedance response is discussed in Hartley et al. U.S. Pat. No. 6,076,015 (“the Hartley et al. patent”) entitled “RATE ADAPTIVE CARDIAC RHYTHM MANAGEMENT DEVICE USING TRANSTHORACIC IMPEDANCE,” assigned to Cardiac Pacemakers, Inc., the disclosure of which is incorporated herein by reference in its entirety.

In one embodiment, the response signal sensed by signal processor 155 is a voltage that, for the given test current, represents a thoracic impedance (i.e., a resistive or reactive impedance associated with at least a portion of a thorax of a living organism). This thoracic impedance signal is influenced by the patient's thoracic intravascular fluid tension, heart beat, and breathing (also referred to as “respiration” or “ventilation”). A “dc” or “baseline” or “low frequency” component of the thoracic impedance signal (e.g., less than a cutoff value that is approximately between 0.1 Hz and 0.5 Hz, inclusive, such as, for example, a cutoff value of approximately 0.1 Hz) provides information about the subject patient's thoracic fluid tension, and is therefore influenced by intravascular fluid shifts to and away from the thorax. Higher frequency components of the thoracic impedance signal are influenced by the patient's breathing (e.g., approximately between 0.05 Hz and 2.0 Hz inclusive) and heartbeat (e.g., approximately between 0.5 Hz and 10 Hz inclusive).

As discussed above, a too-low intravascular fluid tension in the thorax (“thoracic hypotension”) may result from changes in posture. This is sometimes referred to as orthostatic hypotension. For example, in a person who has been in a recumbent position for some time, approximately ⅓ of the blood volume is in the thorax. When that person then sits upright, approximately ⅓ of the blood that was in the thorax migrates to the lower body. This increases thoracic impedance. Approximately 90% of this fluid shift takes place within 2 to 3 minutes after the person sits upright.

Aside from such changes in posture, however, thoracic hypotension may also manifest itself as disautonomic syncope or vasovagal syncope, or other condition in which intravascular fluid shift from the thorax may or may not correspond directly to a change in the patient's posture. According to the present techniques, however, hypotension resulting from a fluid shift away from the thorax is indicated by an increase in the baseline thoracic impedance, regardless of whether the cause of the hypotension is orthostatic. In response to the detection of hypotension, system 100 provides a therapy, such as by increasing the patient's heart rate by delivering pacing stimuli to the heart more rapidly. Upon detecting thoracic hypotension, signal processor 155 outputs an indicated rate signal at node/bus 160 to controller 165. In one embodiment, based on the indicated rate signal at node 160, controller 165 adjusts the rate of delivery of cardiac rhythm management therapy, such as electrical pacing stimuli, to heart 115 by therapy circuit 170. Such pacing stimuli includes, for example, providing bipolar pacing between tip electrode 120 and ring electrode 125, providing unipolar pacing between can electrode 135 and either of tip electrode 120 or ring electrode 125, or providing pacing stimuli using any other suitable electrode configuration. By increasing pacing rate in this manner, system 100 effects a faster return of blood from the extremities to the thorax and head, thereby reducing or avoiding the symptoms of dizziness or fainting. By using thoracic impedance to directly measure thoracic intravascular fluid tension, rather than measuring a change of posture or other secondary variable, system 100 provides more reliable treatment of thoracic hypotension and its associated symptoms. However, it is understood that in one embodiment, system 100 bases its treatment of hypotension based not only on the thoracic impedance-based measurement of intravascular fluid tension, but also one or more of these secondary variables (e.g., also using an accelerometer to detect a change in posture).

In another embodiment, the indicated pacing rate is not based solely on the intravascular thoracic fluid tension information obtained from the thoracic impedance signal. In one such embodiment, for example, the indicated pacing rate is also based on the patient's intrinsic heart rate, such as obtained by sensing intrinsic heart depolarizations using sense amplifier 175. In other embodiments, the intravascular fluid tension information is used in combination with at least one other indication of the patient's metabolic need for an increased heart rate. This other indication of metabolic need is obtained either from the same thoracic impedance sensor technique discussed above (but using other thoracic impedance information such as, for example, breathing or heart rate) or from a completely different sensor. One example of such a different sensor is an accelerometer used to sense the patient's activity as a sensor-driven indicator of the need for a higher heart rate. An example of a “minute ventilation” technique of using the breathing information carried by the thoracic impedance signal as an indication of the patient's metabolic need for a higher heart rate is discussed in Hartley et al. U.S. Pat. No. 6,076,015 (“the Hartley et al. patent”) entitled “RATE ADAPTIVE CARDIAC RHYTHM MANAGEMENT DEVICE USING TRANSTHORACIC IMPEDANCE,” assigned to the assignee of the present invention, the disclosure of which is incorporated herein by reference in its entirety. The background portion of the Hartley et al. patent also discusses a number of other sensors and techniques for providing an indication of metabolic need for adjusting the heart rate; it is understood that such techniques, or any other technique known in the art, could be blended or otherwise used in combination with the techniques discussed herein for increasing heart rate in response to an increase in the baseline transthoracic impedance corresponding to intravascular fluid hypotension. The Hartley et al. patent also discusses in detail one technique for obtaining transthoracic impedance information using exciter 150, however, it is understood that the present invention is not limited to such technique, but could use any known technique for providing a test signal and sensing a resulting impedance.

### Signal Processor

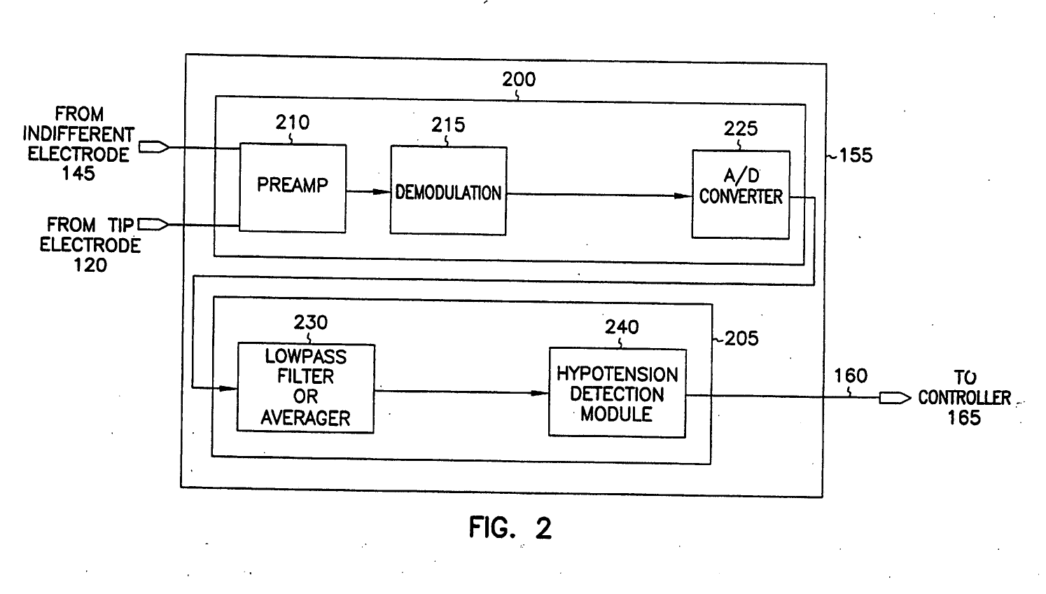

FIG. 2 is a block diagram illustrating generally, by way of example, but not by way of limitation, one embodiment of portions of signal processor 155. In this embodiment, signal processor 155 includes analog signal processing circuit 200 and digital signal processing circuit 205. Inputs of a preamplifier 210 (also referred to as a preamp or a receiver) of analog signal processing circuit 200 are electrically coupled to each of indifferent electrode 145 and tip electrode 120 for receiving a signal in response to the above-discussed stimuli provided by exciter 150. Analog signal processing circuit 200 also includes demodulator 215, receiving the output of preamplifier 210, and providing a demodulated output signal to analog-to-digital (A/D) converter 225. An output signal from A/D converter 225 is received at lowpass filter (or averager) 230 of digital signal processing circuit 205. The Hartley et al. patent discusses the structure and operation of one embodiment of preamp 210 and demodulator 215.

In one embodiment, digital signal processing circuit 205 is included within controller 165 such as, for example, as a sequence of instructions executed by a microprocessor. In another embodiment, digital signal processing circuit 205 includes separately implemented hardware portions dedicated to performing the digital signal processing tasks discussed below. Hypotension detection module 240 receives an output signal from the averager or other lowpass filter 230, and provides an indication of the hypotension at node 160 to controller 165, which controls the rate at which pacing stimuli pulses are issued by therapy circuit 170.

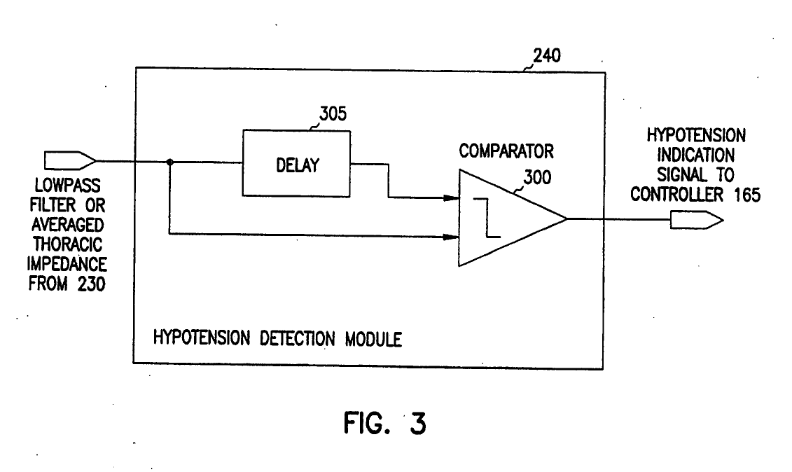

FIG. 3 is a schematic/block diagram illustrating generally, by way of example, but not by way of limitation, one embodiment of portions of hypotension detection module 240. This embodiment provides a digital comparator 300 and a memory storage location, such as delay 305, for storing a previous baseline thoracic impedance received from lowpass filter or averager 230. In one embodiment, this delay is approximately between 3 and 120 seconds inclusive (e.g., 30 seconds). By comparing a difference between the present baseline thoracic impedance and its previous value to a threshold value, comparator 300 provides an output signal that indicates the presence of thoracic hypotension to controller 165. Comparator 300 includes embodiments both with and without hysteresis. In an alternative embodiment, comparator 300 is replaced by a difference circuit that provides, instead of a binary indication of thoracic hypotension, a multi-valued indication of the magnitude of the thoracic hypotension.

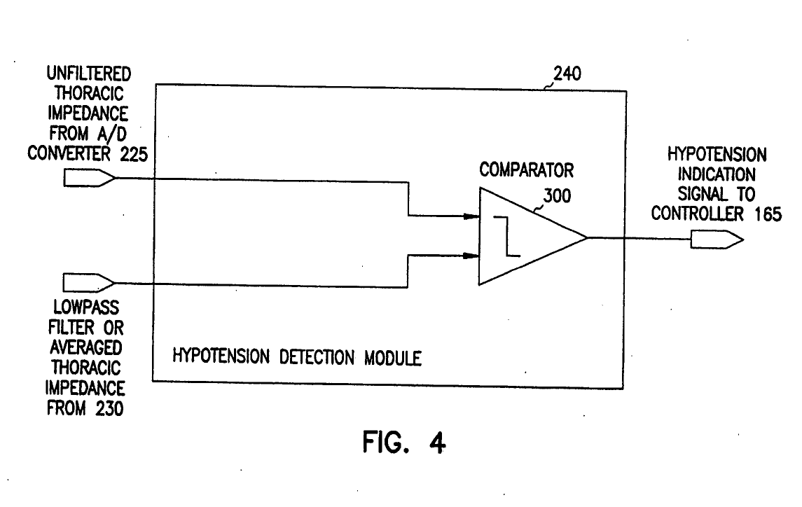

FIG. 4 is a schematic/block diagram illustrating generally, by way of example, but not by way of limitation, another embodiment of portions of hypotension detection module 240. This embodiment includes a digital comparator 300 that receives the baseline thoracic impedance from lowpass filter or averager 230 as well as an unfiltered (or lesser filtered) “instantaneous” thoracic impedance from A/D converter 225. By comparing a difference between the present baseline impedance and the present “instantaneous” thoracic impedance to a threshold value, comparator 300 provides an output signal that indicates the presence of thoracic hypotension to controller 165. Comparator 300 includes embodiments both with and without hysteresis. In an alternative embodiment, comparator 300 is replaced by a difference circuit that provides, instead of a binary indication of thoracic hypotension, a multi-valued indication of the magnitude of the thoracic hypotension. It is understood that the embodiments of FIGS. 3 and 4 may be combined, for example, to base the indication of thoracic hypotension on both the unfiltered and lowpass filtered thoracic impedance signals.

In one embodiment, hypotension detection module 240 indicates the detection of thoracic hypotension based on an increase of approximately between 2 and 20 ohms inclusive (e.g., 6 ohms) in the received lowpass filtered thoracic impedance signal, which, in one embodiment, is averaged such as using a fixed or moving window average over a time period that is approximately between 5 seconds and 5 minutes inclusive (e.g., 1 minute). However, it is understood that lowpass filter 230 need not be an averager; other embodiments include a digital (or, if moved before A/D converter 225, analog) filter having an effective cutoff frequency (i.e., effective 3 dB attenuation point of a single or multiple pole filter) that passes a signal associated with thoracic fluid shift and attenuates a higher frequency signal such as that associated with the patient's breathing or heart beat.

In another embodiment, hypotension detection module 240 also receives a thoracic impedance signal from A/D converter 225, without being filtered or averaged (or alternatively, averaged over a shorter time period) by lowpass filter or averager 230. In this embodiment, hypotension detection module 240 uses this unfiltered thoracic impedance signal in combination with the averaged thoracic impedance signal, output by lowpass filter or averager 230, to indicate the detection of thoracic hypotension. In one example, where this “instantaneous” or “unfiltered” thoracic impedance exceeds the averaged thoracic impedance (also referred to as the “baseline,” “low-frequency,” or “dc” thoracic impedance) by a threshold amount that is approximately between 2 and 30 ohms inclusive, then an indication of thoracic hypotension is triggered. Hypotension detection module 240 provides an indication of detected hypotension, at node 160, to controller 165; this indication may be, among other things, a binary indication of whether hypotension is present or, alternatively, a signal that includes information about the degree of hypotension present (e.g., based on the change in baseline thoracic impedance and/or difference between the instantaneous and average thoracic impedance).

In response to the indication of thoracic hypotension received from hypotension detection module 240, controller 165 provides an appropriate therapy such as by adjusting the rate of delivery of pacing therapy to heart 115. In one embodiment, detection of hypotension triggers pacing at a particular rate programmed by the physician (e.g., 90, 100, 110, 120, 130 beats per minute) for a particular period of time (e.g., 30 seconds to 10 minutes), and then reverts back to the previous pacing rate before hypotension was detected, or to a different pacing rate if so indicated by one or more other sensors. For example, if the patient had a paced or intrinsic heart rate of 60 beats per minute when hypotension is detected, controller 165 then paces the patient at a hypotension treatment rate of 110 beats per minute, for example, to counter the hypotension. After the desired duration of this treatment, such as 2 minutes, for example, controller 165 returns to the previous rate of 60 beats per minute. If, for example, a minute ventilation, accelerometer, or other sensor indicates a metabolic need for a different rate, however (e.g., 70 beats per minute indicated by an accelerometer tracking patient activity) then controller 165 returns to that appropriately indicated rate (e.g., 70 beats per minute indicated by the accelerometer). In a further variation of this and other embodiments, the rate change to or from the hypotension treatment rate is not instantaneous, but incorporates a rate-smoothing algorithm such as is known in the art.

In another embodiment, controller 165 responds to a detection of hypotension by triggering an incremental increase (e.g., 10, 20, 30 . . . 50 beats per minute) in the pacing rate over the previous paced or intrinsic rate for a particular period of time (e.g., 30 seconds to 10 minutes), and then reverts back to that previous pacing rate or other suitable rate such if so determined by other direct or indirect sensors of metabolic need. For example, if hypotension is detected when a patient has a paced or intrinsic heart rate of 65 beats per minute, and an incremental increase of 30 beats per minute has previously been programmed, then controller 165 increases the pacing rate to 95 beats per minute for the programmed duration of this treatment, such as 5 minutes, for example, after which time it reverts back to 65 beats per minute unless a metabolic need sensor indicates that reversion to another rate is more appropriate. In a further embodiment, the incremental increase is itself a function of the intrinsic or sensor-indicated heart rate, so that a higher intrinsic or sensor-indicated rate results in a smaller (or larger) incremental change in pacing rate when hypotension is detected. In yet a further embodiment, the incremental rate increase is a function of the degree of detected hypotension. In such an example, a larger increase in baseline thoracic impedance is associated with a larger incremental rate increase for treating the hypotension.

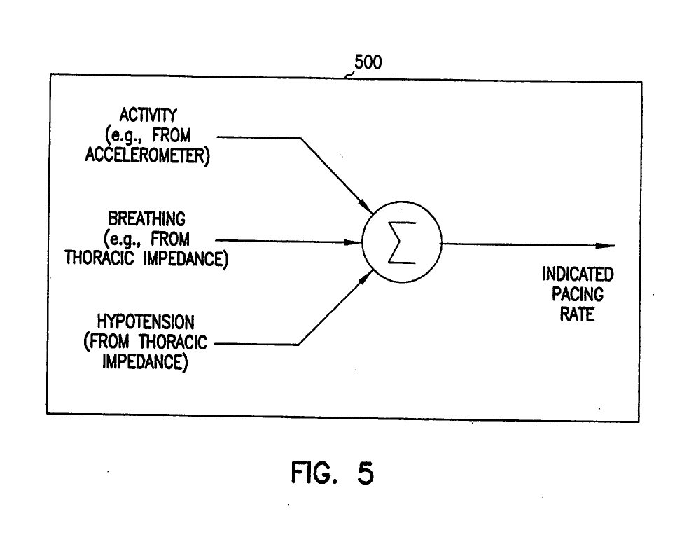

In a further embodiment, hypotension detected based on thoracic impedance is one factor used in providing a blended indicated pacing rate; the indicated pacing rate is also based on at least one other factor such as the patient's breathing (e.g., using a minute ventilation technique) or activity (e.g., using an accelerometer technique). FIG. 5 is a block diagram illustrating generally, by way of example, not by way of limitation, one embodiment of a blending module 500 in controller 165 or elsewhere that provides an indicated pacing rate based on activity, breathing, and/or a thoracic impedance-based measurement of hypotension, each scaled appropriately to provide a desired response.

In yet another embodiment, controller 165 responds to a detection of hypotension by triggering the application of high voltage pacing or other increased electrical stimulation such as, for example, to increase the contractility of the heart. This therapy is carried out either in combination with or in lieu of a rate increase. In one example, a patient being paced using pulses of 2.0 Volts and 0.5 milliseconds is treated, upon detection of hypotension, by pacing pulses at a voltage level approximately between 5 Volts and 50 Volts inclusive, a current level approximately between 5 milliamperes and 200 milliamperes inclusive, and/or a pulsewidth approximately between 10 milliseconds and 100 milliseconds inclusive for a suitable time period (e.g., 1 minute) before returning the therapy to previous energy levels.

In still another embodiment, controller 165 responds to the detection of hypotension by triggering the application of drug therapy from an implantable or external drug delivery system. Thus, the present system envisions a broad range of therapeutic responses to hypotension detected from thoracic impedance.

### Other Electrode Configuration Examples

FIG. 1 illustrated an electrode configuration for measuring transthoracic impedance in which a test current was provided between a ring electrode 125 and a housing electrode 135, a resulting voltage is measured across tip electrode 120 and header electrode 145, and a transthoracic impedance is calculated based on the measured voltage and test current. However, other embodiments use different electrode configurations to determine transthoracic impedance, some examples of which are discussed below.

In a first embodiment of determining transthoracic impedance using the electrodes illustrated in FIG. 1, the test current is provided between the ring electrode 125 and one of housing electrode 135 or header electrode 145, and a resulting voltage is measured across tip electrode 120 and the other of housing electrode 135 or header electrode 145.

In a second embodiment, the test current is provided between tip electrode 120 and one of housing electrode 135 or header electrode 145, and the resulting voltage is measured between ring electrode 125 and the other of housing electrode 135 or header electrode 145.

In a third embodiment, the test current is provided between: (a) one of ring electrode 125 or tip electrode 120; and (b) one of housing electrode 135 or header electrode 145. A resulting voltage is measured across: (a) the same one of ring electrode 125 or tip electrode 120; and (b) and the other of housing electrode 135 or header electrode 145.

In a fourth embodiment, the test current is provided between: (a) one of housing electrode 135 or header electrode 145; and (b) one of tip electrode 120 or ring electrode 125. A resulting voltage is measured across: (a) the same one of housing electrode 135 or header electrode 145; and (b) the other one of tip electrode 120 or ring electrode 125.

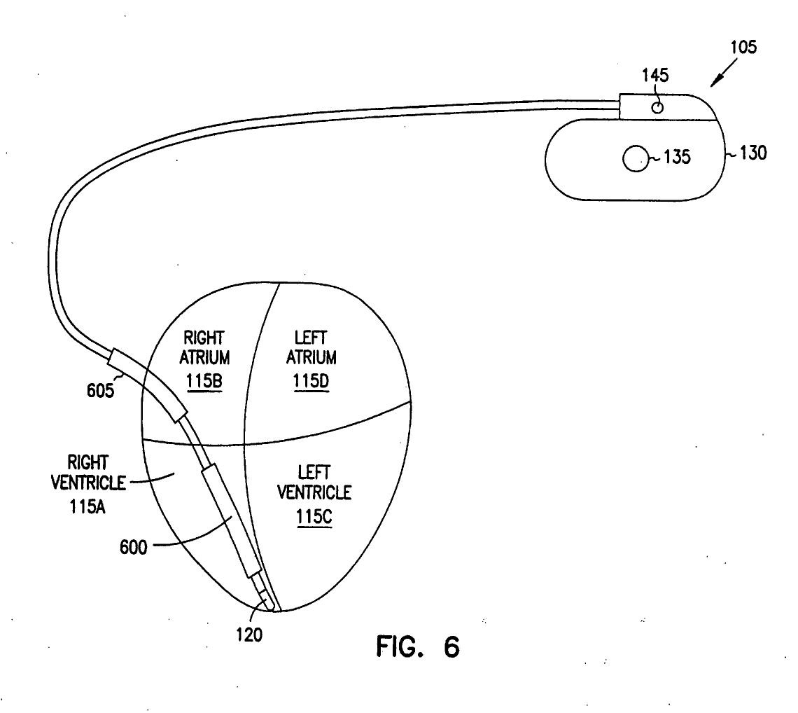

FIG. 6 illustrates generally a cardiac rhythm management device 105 and a lead that includes a tip electrode 120, a first coil or other defibrillation electrode 600 associated with a right ventricle 115A of heart 115, and a second coil or other defibrillation electrode 605 associated with a right atrium 115B of heart 115.

In a first embodiment of determining transthoracic impedance using the electrodes illustrated in FIG. 6, the test current is provided between the first defibrillation electrode 600 and one of housing electrode 135 or header electrode 145, and a resulting voltage is measured across tip electrode 120 and the other of housing electrode 135 or header electrode 145.

In a second embodiment, the test current is provided between tip electrode 120 and one of housing electrode 135 or header electrode 145, and the resulting voltage is measured between first defibrillation electrode 600 and the other of housing electrode 135 or header electrode 145.

In a third embodiment, the test current is provided between: (a) one of first defibrillation electrode 600 or tip electrode 120; and (b) one of housing electrode 135 or header electrode 145. A resulting voltage is measured across: (a) the same one of first defibrillation electrode 600 or tip electrode 120; and (b) and the other of housing electrode 135 or header electrode 145.

In a fourth embodiment, the test current is provided between: (a) one of housing electrode 135 or header electrode 145; and (b) one of tip electrode 120 or first defibrillation electrode 600. A resulting voltage is measured across: (a) the same one of housing electrode 135 or header electrode 145; and (b) the other one of tip electrode 120 or first defibrillation electrode 600.

In a fifth embodiment, the test current is provided between second defibrillation electrode 605 and one of housing electrode 135 or header electrode 145. A resulting voltage is measured across second defibrillation electrode 605 and the other of housing electrode 135 or header electrode 145.

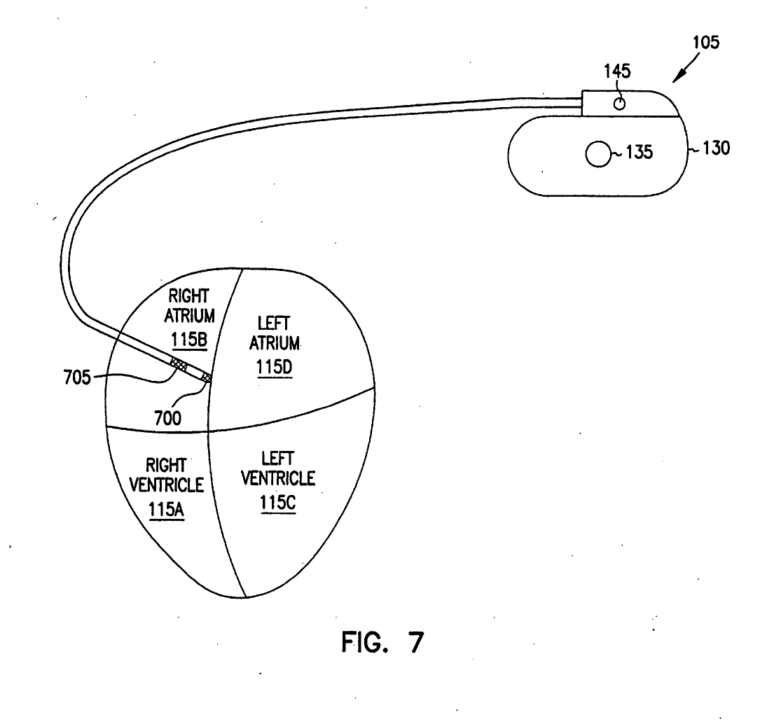

FIG. 7 illustrates generally a cardiac rhythm management device 105 and a lead that includes a tip electrode 700 and ring electrode 705 associated with a right atrium 115B of heart 115.

In a first embodiment of determining transthoracic impedance using the electrodes illustrated in FIG. 7, the test current is provided between the ring electrode 705 and one of housing electrode 135 or header electrode 145, and a resulting voltage is measured across tip electrode 700 and the other of housing electrode 135 or header electrode 145.

In a second embodiment, the test current is provided between tip electrode 700 and one of housing electrode 135 or header electrode 145, and the resulting voltage is measured between ring electrode 705 and the other of housing electrode 135 or header electrode 145.

In a third embodiment, the test current is provided between: (a) one of ring electrode 705 or tip electrode 700; and (b) one of housing electrode 135 or header electrode 145. A resulting voltage is measured across: (a) the same one of ring electrode 705 or tip electrode 700; and (b) and the other of housing electrode 135 or header electrode 145.

In a fourth embodiment, the test current is provided between: (a) one of housing electrode 135 or header electrode 145; and (b) one of tip electrode 700 or ring electrode 705. A resulting voltage is measured across: (a) the same one of housing electrode 135 or header electrode 145; and (b) the other one of tip electrode 700 or ring electrode 705.

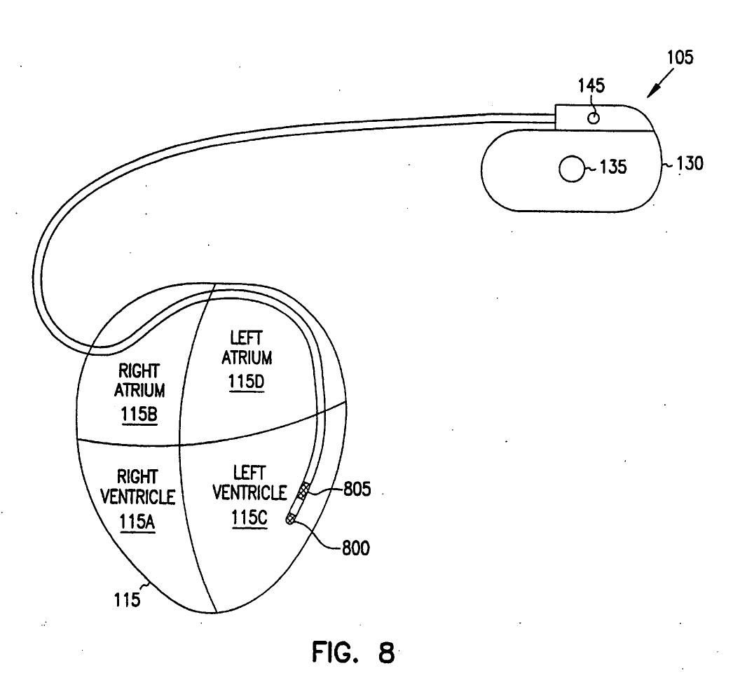

FIG. 8 illustrates generally a cardiac rhythm management device 105 and a lead that includes a tip electrode 800 and ring electrode 805 associated with a left ventricle 115C of heart 115 by being introduced into a coronary sinus and/or great cardiac vein and/or one of its tributaries in the left ventricular free wall, or by being epicardially placed in proximity to left ventricle 115C.

In a first embodiment of determining transthoracic impedance using the electrodes illustrated in FIG. 8, the test current is provided between the ring electrode 805 and one of housing electrode 135 or header electrode 145, and a resulting voltage is measured across tip electrode 800 and the other of housing electrode 135 or header electrode 145.

In a second embodiment, the test current is provided between tip electrode 800 and one of housing electrode 135 or header electrode 145, and the resulting voltage is measured between ring electrode 805 and the other of housing electrode 135 or header electrode 145.

In a third embodiment, the test current is provided between: (a) one of ring electrode 805 or tip electrode 800; and (b) one of housing electrode 135 or header electrode 145. A resulting voltage is measured across: (a) the same one of ring electrode 805 or tip electrode 800; and (b) and the other of housing electrode 135 or header electrode 145.

In a fourth embodiment, the test current is provided between: (a) one of housing electrode 135 or header electrode 145; and (b) one of tip electrode 800 or ring electrode 805. A resulting voltage is measured across: (a) the same one of housing electrode 135 or header electrode 145; and (b) the other one of tip electrode 800 or ring electrode 805.

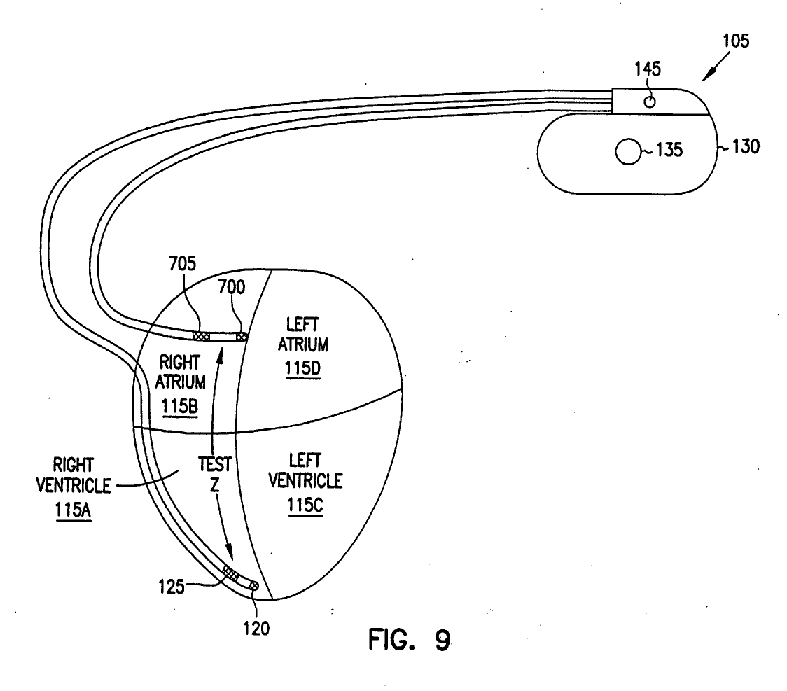

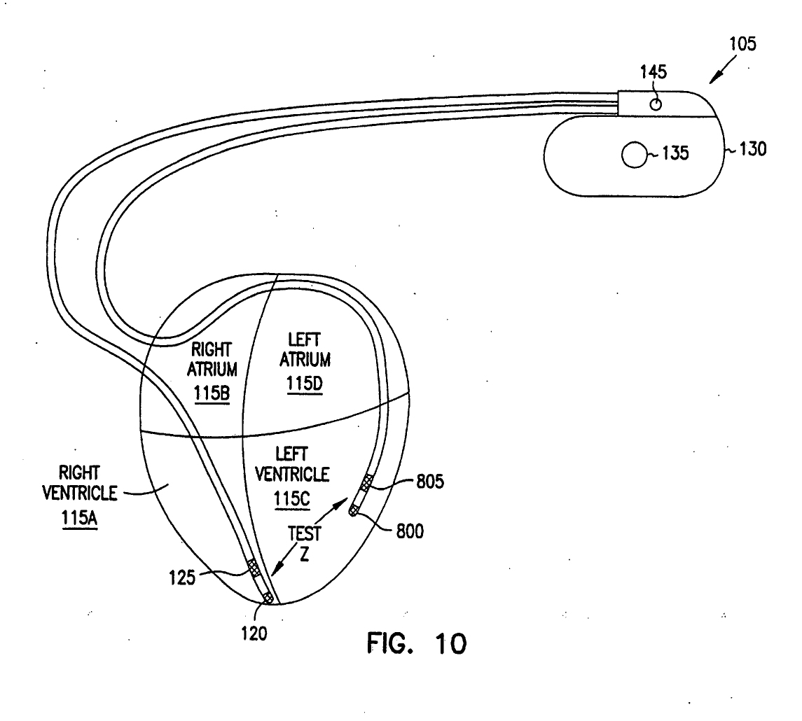

FIGS. 1 and 6-8 illustrate, among other things, configurations in which one or more electrodes is located inside of, or in close proximity to, heart 115, and one or more electrodes is located at a distance away from heart 115, such as, for example, on pectorally or abdominally implanted device 105. This distance provides an effective portion of the thorax for which the transthoracic impedance is sampled. However, a lesser distance may also be used for sampling thoracic impedance, in fact, thoracic impedance may be sampled using electrodes in more close proximity, such as an embodiment in which all electrodes are associated with heart 115, as illustrated in the examples of FIGS. 9 and 10.

Some of the embodiments discussed above (e.g., that of FIG. 1) use four electrodes for measuring impedance. In such a technique for measuring impedance, both electrodes used for sensing the voltage are different than both of the electrodes used for delivering the test current, so that the impedance of the leads coupling the test current source to the test current electrodes does not affect the voltage measurement. However, this is not a requirement. A three electrode arrangement (in which only one of the voltage sensing electrodes is different from the test current electrode pair) or even a two electrode arrangement (using the same electrodes for both delivering the test current and measuring the test voltage) may be used, although such arrangements may result in a larger steady-state (i.e., offset) component of the measured impedance due to the measurement apparatus and unrelated to the steady-state thoracic tissue impedance. The above-discussed system provides, among other things, a cardiac rhythm management system using thoracic impedance measurements to detect and treat hypotension. It detects and treats hypotension that results either from a change in posture or is independent of any change in posture.

### EXAMPLE 2

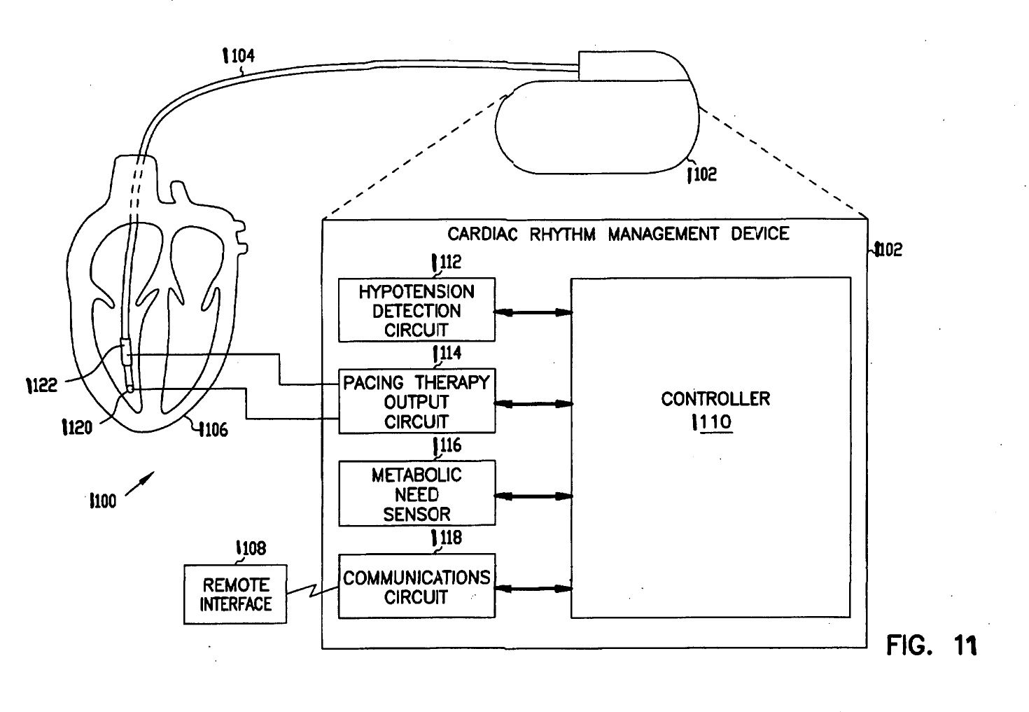

FIG. 11 is a schematic/block diagram example of portions of a cardiac rhythm management system 1100 and portions of an environment in which it is used. In this example, system 1100 includes, among other things, a cardiac rhythm management device 1102 and leadwire (“lead”) 1104, which is coupled to device 1102 for communicating one or more signals between device 1102 and a portion of a living organism or other subject, such as heart 1106. Examples of device 1102 include, among other things, bradycardia and antitachycardia pacemakers, cardioverters, defibrillators, combination pacemaker/defibrillators, drug delivery devices, and any other implantable or external cardiac rhythm management apparatus capable of providing therapy to heart 1106. System 1100 may also include additional components such as, for example, an external or other remote interface 1108 capable of communicating with device 1102.

In this example, device 1102 includes, among other things, a microprocessor or other controller 1110 coupled to a hypotension detection circuit 1112, a pacing therapy output circuit 1114, a metabolic need sensor 1116, and a communication circuit 1118. Communication circuit 1118 is adapted for wireless communication with remote interface 1108. Pacing therapy output circuit 1114 is coupled to one or more electrodes associated with any chamber(s) of heart 1106, such as electrodes 1120 and 1122 of lead 1104, for delivering electrical pacing stimulations for evoking responsive heart contractions. Metabolic need sensor 1116 senses the subject's need for a particular degree of cardiac output of blood being pumped through the subject's circulatory system. To accommodate the sensed metabolic need, controller 1110 provides pacing therapy output circuit 1114 with a variable indicated pacing rate for evoking the heart contractions. A higher sensed metabolic need for cardiac output results in a higher indicated pacing rate for evoking heart contractions.

In this example, hypotension detection circuit 1112 detects a hypotension condition in the subject. In response to the detected hypotension, controller 1110 adjusts the indicated pacing rate. More particularly, in the presence of hypotension, controller 1110 increases a rate response factor (“RRF”) so that a particular degree of metabolic need results in an at least temporarily higher indicated pacing rate than if hypotension were not detected. In a further example, controller 1110 communicates an indication of the hypotension condition through communication circuit 1118 to remote interface 1108 for display or other user output.

One example of metabolic need sensor 1116 is an activity sensor that senses the subject's activity. A greater activity level corresponds to a greater metabolic need for cardiac output of blood pumped through the circulatory system. One particular example of an activity sensor is an accelerometer for sensing the subject's movement, which is deemed correlative to the subject's activity and, therefore, to the subject's metabolic need. One suitable example of an accelerometer-based activity sensor of metabolic need is discussed in Meyerson et al. U.S. Pat. No. 5,179,947 entitled “ACCELERATION-SENSITIVE CARDIAC PACEMAKER AND METHOD OF OPERATION,” which is assigned to Cardiac Pacemakers, Inc., and the disclosure of which is incorporated herein by reference in its entirety. Another example of an activity sensor is a breathing (or “respiration” or “ventilation”) sensor that senses the subject's breathing rate. A higher breathing rate is deemed to correspond to a higher activity level, which, in turn, corresponds to a greater metabolic need.

One particular example of a respiration sensor is a transthoracic impedance sensor that detects an impedance across a portion of a subject's thorax (“thoracic impedance” or “transthoracic impedance.”) In this document, the term “thorax” refers to the subject's body other than the subject's head, arms, and legs. As the subject breathes, inhaling and exhaling (also referred to as inspiration and expiration) the thoracic impedance varies as modulated by the breathing. From these thoracic impedance variations, the breathing rate can be determined.

In such an thoracic impedance respiration sensor example, metabolic need sensor 1116 is coupled to the patient's thorax by at least two electrodes for determining the thoracic impedance by providing a test signal and measuring a response signal. In one suitable thoracic impedance respiration sensor example, system 1100 includes a configuration of at least four electrodes for detecting thoracic impedance, such as discussed in Hauck et al. U.S. Pat. No. 5,284,136 entitled “DUAL INDIFFERENT ELECTRODE PACEMAKER,” assigned to Cardiac Pacemakers, Inc., the disclosure of which is incorporated herein by reference in its entirety. However, a different number of electrodes (e.g., 2 or 3 electrodes, or more than 4 electrodes) could also be used. One suitable example of a metabolic need sensor 1116 based on thoracic impedance detection of respiration uses a high frequency carrier signal to provide a test stimulus and obtain a thoracic impedance response, as discussed in Hartley et al. U.S. Pat. No. 6,076,015 (“the Hartley et al. patent”) entitled “RATE ADAPTIVE CARDIAC RHYTHM MANAGEMENT DEVICE USING TRANSTHORACIC IMPEDANCE,” assigned to Cardiac Pacemakers, Inc., the disclosure of which is incorporated herein by reference in its entirety.

In this example, hypotension detection circuit 1112 detects a hypotension condition in the subject. One example of a suitable hypotension detection circuit 1112 is discussed above in Example 1.

The thoracic impedance signal is influenced by the patient's thoracic intravascular fluid tension, heart beat, and breathing (also referred to as “respiration” or “ventilation”). A “dc” or “baseline” or “low frequency” component of the thoracic impedance signal (e.g., less than a cutoff value that is approximately between 0.1 Hz and 0.5 Hz, inclusive, such as, for example, a cutoff value of approximately 0.1 Hz) provides information about the subject patient's thoracic fluid tension, and is therefore influenced by intravascular fluid shifts to and away from the thorax. Higher frequency components of the thoracic impedance signal are influenced by the patient's breathing (e.g., approximately between 0.05 Hz and 2.0 Hz inclusive) and heartbeat (e.g., approximately between 0.5 Hz and 10 Hz inclusive).

As discussed above, a too-low intravascular fluid tension in the thorax (“thoracic hypotension”) may result from changes in posture. This is sometimes referred to as orthostatic hypotension. For example, in a person who has been in a recumbent position for some time, approximately ⅓ of the blood volume is in the thorax. When that person then sits upright, approximately ⅓ of the blood that was in the thorax migrates to the lower body. This increases thoracic impedance. Approximately 90% of this fluid shift takes place within 2 to 3 minutes after the person sits upright.

Aside from such changes in posture, however, thoracic hypotension may also manifest itself as disautonomic syncope or vasovagal syncope, or other condition in which intravascular fluid shift from the thorax may or may not correspond directly to a change in the patient's posture. However, hypotension resulting from a fluid shift away from the thorax is indicated by an increase in the baseline thoracic impedance, regardless of whether the cause of the hypotension is orthostatic. In response to the detection of hypotension, controller 1110 increases the rate response factor relating the degree of metabolic need sensed by metabolic need sensor 1116 to the indicated pacing rate at which pacing stimulations are provided by pacing output therapy circuit 1114.

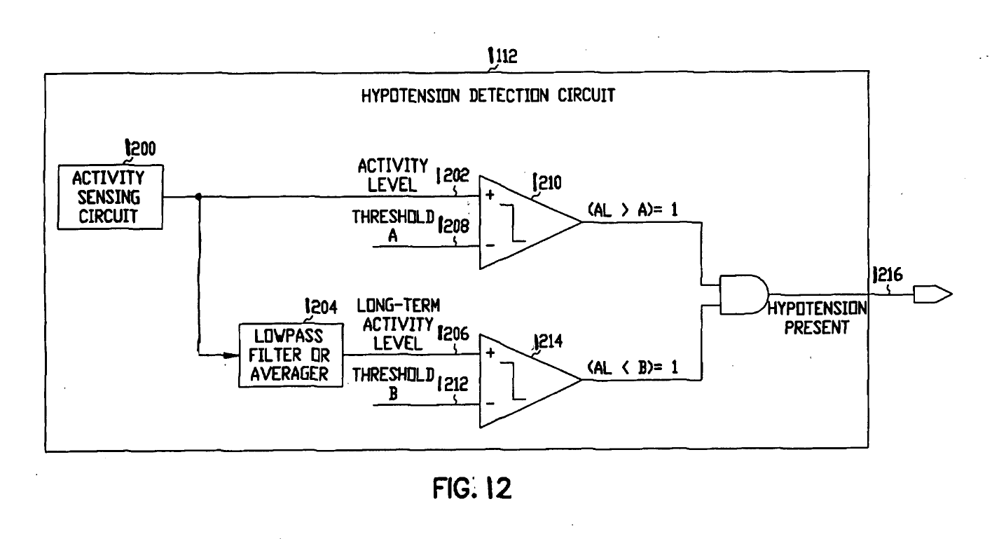

FIG. 12 is a block diagram example of another hypotension detection circuit 1112 using an activity sensing circuit 1200. In one example, activity sensing circuit 1200 includes an accelerometer circuit sensing the subject's motion, which is deemed correlative to the subject's activity, and providing at node 1202 a resulting substantially instantaneous activity level (AL) output signal indicative of the subject's activity. In another example, activity sensing circuit 1200 includes a respiration circuit (using the thoracic impedance technique discussed above or any other suitable technique for detecting a breathing rate) sensing the subject's breathing rate, which is deemed correlative to the subject's activity, and providing at node 1202 a resulting substantially instantaneous AL output signal indicative of the subject's activity. An input of lowpass filter (or averager) 1204 is coupled to receive the substantially instantaneous AL signal for lowpass filtering or averaging over an extended period of time, such as approximately between 15 minutes and 24 hours. Filter 1204 outputs at node 1206 a resulting long-term AL signal. The substantially instantaneous AL at node 1202 is compared to a threshold value A at node 1208 by comparator 1210. The long-term AL at node 1206 is compared to a threshold value B at node 1212 by comparator 1214. If the substantially instantaneous AL exceeds threshold A and the threshold B exceeds the long-term AL, then the subject is deemed to have transitioned from a period of rest to a period of activity. This detected transition is, in turn, deemed to correspond to an onset of orthostatic hypotension as communicated by the output of hypotension detection circuit, at node 1216, to controller 1110. The signal processing illustrated in FIG. 2 can be performed in either analog or digital domains.

Although hypotension detection circuit 1112 and metabolic need sensor 1116 are illustrated in FIG. 11 as being implemented separately, in certain examples these blocks may share certain components. For example, where an accelerometer is used as metabolic need sensor 1116 and hypotension detection circuit 1112 also uses an accelerometer-based activity sensing circuit 1200, the same accelerometer can be used for both. Similarly, where thoracic impedance sensing of breathing is used as metabolic need sensor 1116 and hypotension detection circuit 1112 uses a thoracic impedance baseline for determining whether hypotension is present, the same test signal generation, receiving, and demodulation circuit could be used in both blocks, with appropriate separate processing of different frequency components of the thoracic impedance signal.

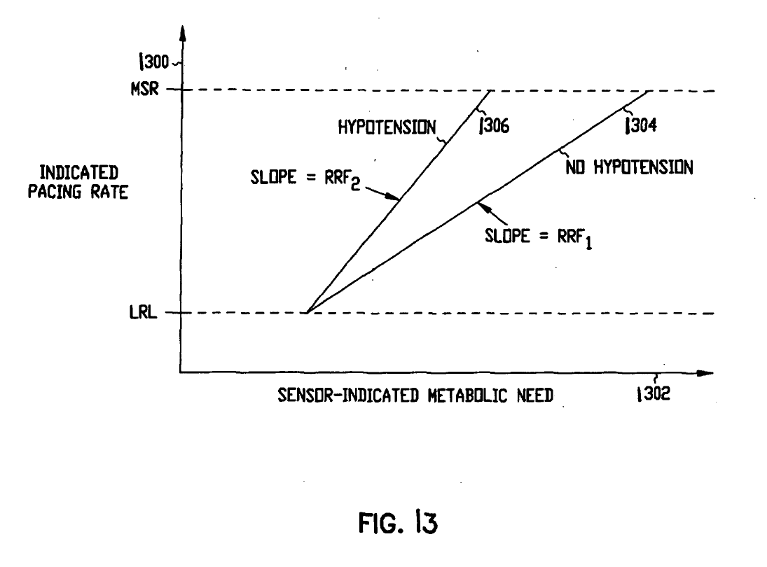

FIG. 13 is a graph example of one technique executed by controller 1110 for determining the indicated pacing rate 1300 from the sensor-indicated metabolic need 1302 received by controller 1110 from metabolic need sensor 1316. In this example, line 1304 indicates one mapping of metabolic need to the indicated pacing rate, which is bounded by a lower rate limit (LRL) and a maximum sensor rate (MSR). A greater metabolic need corresponds to a higher indicated pacing rate, therefore line 1304 has a positive slope. The slope of line 1304 is referred to as the rate response factor (RRF). The RRF is typically programmable to a particular value within a range of values. In operation, upon receiving an indication of a detected episode of hypotension from hypotension detection circuit 1112, controller 1110 increases the RRF from its programmed value, RRF₁, to a higher value, RRF₂, for a time period following the detection of hypotension, and then returns to RRF₁. In one example, this time period is approximately between 30 seconds and 10 minutes, such as about 2 minutes. During this time period, line 1306 illustrates the mapping of metabolic need to indicated pacing rate. Thus, when hypotension is detected, a particular level of sensor-indicated metabolic need results in a higher value of the indicated pacing rate than when no hypotension is present. Controller 1110 provides the indicated pacing rate to pacing therapy output circuit 1114, which, in turn, provides pacing stimuli to heart 1106 at the indicated pacing rate.

For example, when activity is used to indicate metabolic need, when hypotension is detected the indicated pacing rate is increased. The increase in indicated pacing rate is larger at higher activity levels than at lower activity levels. By increasing the indicated pacing rate in this manner, controller 1110 effects a faster return of blood from the extremities to the thorax and head, thereby reducing or avoiding the symptoms of dizziness or fainting.

In an alternative example, rather than abruptly being stepped back from RRF₂ to RRF₁ following the time period initiated by the detected hypotension, the mapping slope more slowly decays, or otherwise incrementally steps back to the programmed value. In one example, the RRF approximately exponentially decays from RRF₂ to RRF₁, such as with a time constant that is approximately between 15 seconds and 10 minutes, such as about 1 minute. In another example, the RRF incrementally steps from RRF₂ to RRF₁ through a number of intermediate values that are substantially equally spaced between RRF₂ and RRF₁.

### EXAMPLE 3

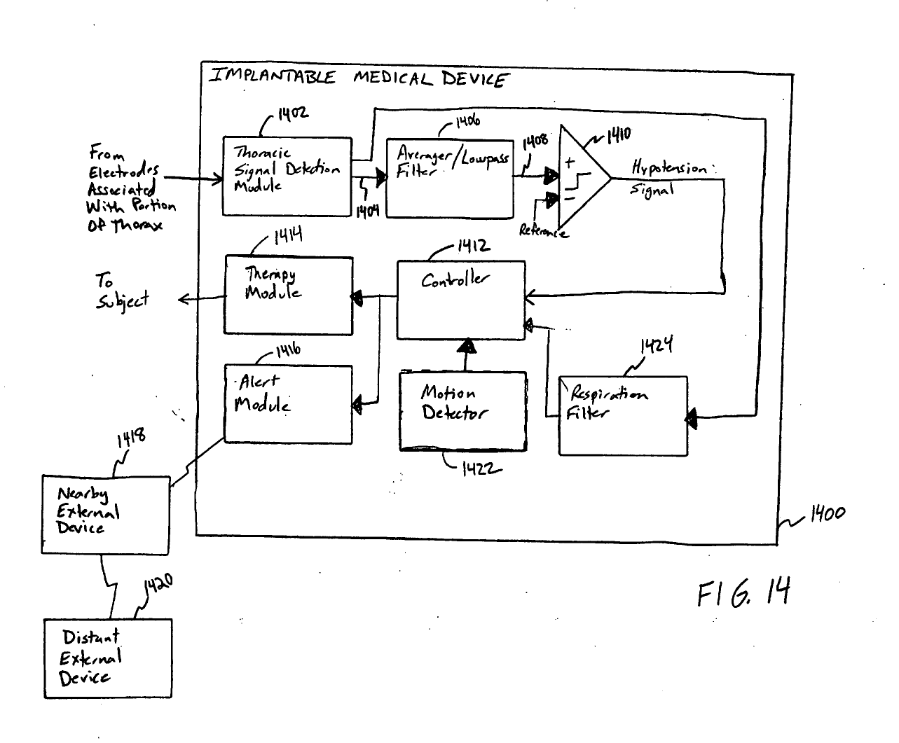

FIG. 14 is a block diagram illustrating generally portions of an implantable medical device 1400 that receives a thoracic impedance signal from electrodes associated with a portion of a subject's thorax. The thoracic impedance signal is received at thoracic signal detection module 1402. In this example, the resulting detected thoracic signal output at 1404 is received at an input of the averager/lowpass filter 1406, which attenuates frequency components of the detected thoracic impedance signal that are not indicative of hypotension (e.g., respiration, cardiac stroke components), and which passes or amplifies frequency components of the detected thoracic impedance signal that are indicative of hypotension. The resulting signal is output at node 1408 to a comparator circuit 1410, where it is compared to a reference. The comparator circuit 1410 outputs a resulting signal indicating whether or the degree to which hypotension is present.

This hypotension signal is received by a controller circuit 1412, which, in one example is coupled to either a therapy module 1414 or an alert module 1416, or both. In one example, when the hypotension signal indicates that hypotension is present, the alert module 1416 provides a responsive alert. The responsive alert can be delivered to the subject, such as by using a vibrator, electrical-to-audible transducer, or other like device included in the alert module 1416. In another example, the alert module 1416 includes a telemetry circuit to deliver a responsive alert from the implantable medical device 1400, instead of or in addition to delivering therapy. The alert can be communicated to a nearby external device 1418, such as an associated programmer, or the like. In one example, the alert is then further communicated to a distant external device 1420, such as using a computer, telephony, or other communications network. This permits alerting a remote caregiver to the hypotension condition, or logging of the hypotension condition at an external patient database. If the alert module 1416 includes a long-range communications device, it can then communicate with the distant external device 1420 directly and, in one example, the nearby external device 1418 can be omitted.

In one example, the therapy module 1414 includes a pacing circuit for delivering or adjusting pacing therapy when hypotension is present, as discussed in the above examples. In another example, the therapy module 1414 includes an infusion dispenser or drug delivery device for delivering or adjusting delivery of a drug or other substance, such as when hypotension is present, or based on a degree of hypotension present, as also discussed in the above examples.

In one example, the therapy module includes an autonomic nervous system stimulation device, such as to stimulate the sympathetic nervous system or inhibit the parasympathetic nervous system, as discussed in Libbus et al. U.S. patent application Ser. No. 11/124,791 (Attorney Docket 279.780US1), entitled METHOD AND APPARATUS FOR CONTROLLING AUTONOMIC BALANCE USING NEURAL STIMULATION, filed on May 9, 2005, which is assigned to Cardiac Pacemakers, Inc., and which is incorporated herein by reference in its entirety, including its description of controlling autonomic balance using neural stimulation. In another example, such stimulation is delivered to pain receptors that encourage production of adrenaline or the like to enhance circulation and return fluid to the thorax.

In another example, the therapy module includes an autonomic nervous system stimulation device, such as to provide or control gene therapy to treat hypotension, such as by enhancing circulation or otherwise assisting in returning fluid to the thorax, such as discussed in Girouard et al. U.S. patent application Ser. No. 10/788,906 (Attorney Docket No. 279.696US1) entitled METHOD AND APPARATUS FOR DEVICE CONTROLLED GENE EXPRESSION, filed on Feb. 27, 2004, and which is assigned to Cardiac Pacemakers, Inc., and which is incorporated herein by reference in its entirety, including its description of providing or controlling gene therapy such as cell delivery or the like.

The controller need not activate the alert or the therapy based solely on hypotension. In one example, the alert or therapy also is activated based on at least one other factor such as motion (as determined by an accelerometer or other motion detector circuit 1422) or breathing (as determined by a respiration filter 1424 that extracts a respiration signal from the thoracic impedance signal, as also discussed in the above examples.

### CONCLUSION

It is to be understood that the above description is intended to be illustrative, and not restrictive. For example, the above-described embodiments (and/or aspects thereof) may be used in combination with each other. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The Abstract of the Disclosure is provided to comply with 37 C.F.R. § 1.72(b), requiring an abstract that will allow the reader to quickly ascertain the nature of the technical disclosure. It is submitted with the understanding that it will not be used to interpret or limit the scope or meaning of the claims. In addition, in the foregoing Detailed Description, various features may be grouped together to streamline the disclosure. This method of disclosure is not to be interpreted as reflecting an intention that the claimed embodiments require more features than are expressly recited in each claim. Rather, as the following claims reflect, inventive subject matter may lie in less than all features of a single disclosed embodiment. Thus the following claims are hereby incorporated into the Detailed Description, with each claim standing on its own as a separate embodiment.

1. A machine-assisted method including:
detecting a thoracic impedance signal associated with a portion of a subject's thorax; and
providing a therapy at least in part in response to a baseline portion of the detected thoracic impedance below about 0.5 Hz indicating a fluid shift away from the thorax, the therapy assisting to shift fluid back toward the thorax.

2. The method of claim 1, in which the providing the therapy includes providing a non-pacing therapy.

3. The method of claim 1, in which the providing the therapy includes providing a non-pacing therapy and a pacing therapy.

4. The method of claim 2, in which the providing the non-pacing therapy includes stimulating an autonomic nervous system.

5. The method of claim 2, in which the providing the non-pacing therapy includes providing or controlling a gene therapy.

6. The method of claim 2, in which the providing the non-pacing therapy includes delivering a drug.

7. The method of claim 1, further including detecting a motion of the subject and providing the therapy based at least in part on the detected motion of the subject.

8. The method of claim 1, further including detecting a breathing of the subject and providing the therapy based at least in part on the detected breathing.

9. The method of claim 1, in which providing the therapy includes adjusting the therapy based on frequency components of the thoracic impedance associated with fluid shift away from the thorax and associated with the subject's breathing.

10. The method of claim 1, in which providing the therapy includes providing a drug to the subject in response to an increase in detected thoracic impedance at a frequency associated with fluid shift away from the thorax.

11. The method of claim 1, in which the detecting the thoracic impedance signal includes detecting a thoracic fluid shift signal having a frequency component that is less than or equal to a cutoff frequency value that is between 0.01 Hz and 0.5 Hz inclusive.

12. The method of claim 1, in which the providing the therapy includes treating both a hypotension associated with a change in a subject's posture and a hypotension that is not associated with a change in the subject's posture.

13. An implantable medical device, configured to utilize first and second electrodes associated with a portion of a subject's thorax, the implantable medical device comprising:
a thoracic signal detection module, for coupling to the first and second electrodes to receive a thoracic impedance signal;
an averager/lowpass filter that obtains a baseline portion of the thoracic impedance signal below about 0.5 Hz that is associated with a fluid shift away from the thorax; and
a therapy module to provide therapy to the subject using the baseline portion of the thoracic impedance signal, the therapy assisting to shift fluid back toward the thorax.

14. The device of claim 13, in which the averager/lowpass filter attenuates a breathing portion of the thoracic impedance signal, and in which the lowpass filter attenuates a cardiac stroke portion of the thoracic impedance signal.

15. The device of claim 13, in which the therapy module includes an autonomic nervous system stimulation circuit.

16. The device of claim 13, in which the therapy module includes a pacing circuit.

17. The device of claim 13, in which the therapy module includes an infusion dispenser.

18. The device of claim 13, in which the therapy module includes a gene therapy controller.

19. The device of claim 13, further including a motion detector circuit, and in which the therapy module provides the therapy based at least in part on the detected motion of the subject.

20. The device of claim 13, further including a respiration filter, coupled to the thoracic signal detection module to detect a breathing of the subject, and in which the therapy module is coupled to the respiration filter, and in which the therapy module provides the therapy based at least in part on the detected breathing.

21. A machine-assisted method including:
detecting a thoracic impedance signal associated with a portion of a subject's thorax; and
providing an alert at least in part in response to a baseline portion of the detected thoracic impedance below about 0.5 Hz indicating a fluid shift away from the thorax, the therapy assisting to shift fluid back toward the thorax.

22. The method of claim 21, in which the providing the alert includes communicating from an implantable device to an external device.

23. The method of claim 21, in which the providing the alert includes communicating from an implantable device to a nearby first external device and communicating from the nearby first external device to an allowably distant second external device.

24. The method of claim 21, in which the providing the alert includes providing the alert to be discernable to the subject.

25. The method of claim 21, comprising providing a therapy at least in part in response to a baseline portion of the detected thoracic impedance below about 0.5 Hz indicating a fluid shift away from the thorax, the therapy assisting to shift fluid back toward the thorax.

26. The method of claim 25, in which the providing the therapy includes providing a pacing therapy.

27. The method of claim 25, in which the providing the therapy includes providing a non-pacing therapy.

28. The method of claim 25, in which the providing the therapy includes stimulating an autonomic nervous system.

29. The method of claim 25, in which the providing the therapy includes providing a drug.

30. The method of claim 25, in which the providing the non-pacing therapy includes providing a gene therapy.

31. The method of claim 21, including detecting a motion of the subject and providing the alert based at least in part on the detected motion of the subject.

32. The method of claim 21, including:
logging at least one hypotension episode; and
communicating information about the at least one hypotension episode.

33. An implantable medical device, configured to utilize first and second electrodes associated with a portion of a subject's thorax, the implantable medical device comprising:
a thoracic signal detection module, for coupling to the first and second electrodes to receive a thoracic impedance signal;
an averager/lowpass filter that obtains a baseline portion of the thoracic impedance signal below about 0.5 Hz that is associated with a fluid shift away from the thorax; and
an alert module to provide an alert using the baseline portion of the thoracic impedance signal indicating a hypotension episode in which fluid shifts away from the thorax.

34. The device of claim 33, in which the alert module includes a telemetry circuit to communicate the alert from the implantable medical device to an external device.

35. The device of claim 33, in which the alert module includes a vibrator.

36. The device of claim 33, in which the alert module includes an electrical-to-audible signal transducer.

37. The device of claim 33, including a pacing circuit to deliver or adjust pacing therapy in response to the thoracic impedance signal indicating a fluid shift away from the thorax.

38. The device of claim 33, including a non-pacing therapy module to deliver or adjust therapy in response to the thoracic impedance signal indicating a fluid shift away from the thorax.

39. The device of claim 38, in which the non-pacing therapy module includes an autonomic nervous system stimulator.

40. The device of claim 38, in which the non-pacing therapy module includes an infusion dispenser.
